(19) United States
(12) Patent Application Publication
(10) Pub. No.: US 2021/0391079 A1
(43) Pub. Date: Dec. 16, 2021

(54) METHOD AND APPARATUS FOR MONITORING A PATIENT

(71) Applicant: Oxford University Innovation Limited, Oxford (Oxfordshire) (GB)

(72) Inventors: David CLIFTON, Oxford (Oxfordshire) (GB); Tingting ZHU, Oxford (Oxfordshire) (GB); Thomas TAYLOR, Uxbridge (Middlesex) (GB); Hamza JAVED, Oxford (Oxfordshire) (GB); Rasheed EL-BOURI, Swansea (South Wales) (GB); Iain DUNN, Reading (Berkshire) (GB); Peter WATKINSON, Oxford (Oxfordshire) (GB); Jennifer BISHOP, Oxford (Oxfordshire) (GB)

(21) Appl. No.: 17/285,535

(22) PCT Filed: Sep. 23, 2019
(86) PCT No.: PCT/GB2019/052662

§ 371 Date: Apr. 15, 2021

(30) Foreign Application Priority Data
Oct. 30, 2018 (GB) 1817708.9

(51) Int. Cl. G16H 50/20; G16H 10/60; G16H 50/70; G16H 40/20; G16H 40/67; G06N 20/20; A61B 5/00

(52) U.S. Cl. CPC G16H 50/20; G16H 10/60; G16H 50/70; A61B 5/7275; G16H 40/67; G06N 20/20; A61B 5/7267; G16H 40/20

(57) ABSTRACT

Methods and apparatus for monitoring a patient are provided. In one arrangement, a multi- dimensional patient data set is received at each of a plurality of different reference times. Each dimension of the patient data set stores a value representing a different type of information about the patient. A plurality of predictions of a health trajectory of the patient are generated. Each prediction is generated using a trained machine learning model receiving as input a different one of the patient data sets. The trained machine learning model may be dimensionally adaptive, such that predictions of the patient trajectories are provided using patient data sets having different respective dimensionalities for at least a sub-set of the reference times. The trained machine learning model may use machine learned predictions of accuracy to select trained machine learning units from an ensemble of trained machine learning units.

The present invention relates to monitoring a patient, particularly for the purpose of supporting control of patient flow in a medical facility such as a hospital.

Patient flow is a term used to describe the ease with which a patient moves through different stages of a medical facility, e.g. without being subject to or causing delays. Increasing demands on healthcare systems due to factors such as population growth and ageing are having a negative impact on patient flow. Current approaches to managing this problem are often reactive, resulting in less than optimal performance and significant delays in a patient's care and/or eventual discharge. This problem particularly centres around the Emergency Department (ED), where timescales are shorter, and decisions need to be made quickly, often before all the variables can be considered.

It is an object of the invention to provide methods and apparatus for monitoring patients that support improved control of patient flow.

According to an aspect of the invention, there is provided a computer-implemented method of monitoring a patient, comprising: receiving a multi-dimensional patient data set at each of a plurality of different reference times, each dimension of the patient data set storing a value representing a different type of information about the patient; and generating a plurality of predictions of a health trajectory of the patient, each prediction being generated using a trained machine learning model receiving as input a different one of the patient data sets, wherein the trained machine learning model is dimensionally adaptive, such that predictions of the patient trajectories are provided using patient data sets having different respective dimensionalities for at least a sub-set of the reference times.

Thus, a method is provided in which a patient is monitored using a trained machine learning model that is able to adapt actively to changes in availability of information as a function of time. In contrast to alternative approaches that use a fixed trained machine learning model to make predictions at a fixed point in time, the current approach has been found to provide improved flexibility, accuracy and reliability. Models used in the alternative approaches may perform well in the situation they are trained on, but may not be optimal at a different time when new information has become available or where other changes to the situation have occurred. They are therefore not robust to be used in real-life applications over a prolonged period of time.

In some embodiments, the trained machine learning model switches between different trained machine learning units of an ensemble of trained machine learning units, the switching being controlled for each patient data set based on a dimensionality of the patient data set. Thus, the trained machine learning model can adapt to changing availability of information by switching between trained machine learning units capable of receiving input data sets of different dimensionalities. For example, at a reference time where one or more new types of information have become available since an immediately preceding reference time, the trained machine learning model may switch between use of a first trained machine learning unit that has not been trained to use the one or more new types of information to a second trained machine learning unit that has been trained to use the one or more new types of information. In a case where new types of information are progressively made available over time, the trained machine learning model may progressively transition to architectures (trained machine learning units) adapted to handle problems with higher dimensionality feature sets. As a patient's treatment progresses, more sophisticated machine learning units may thus be considered in an incremental fashion as more information about the patient becomes available. Alternatively or additionally, progressive transition may be made to trained machine learning units capable of processing different information. Thus, the machine learning architecture is progressively adapted in time so as to continually be able to provide optimized predictions even as the nature of available information, and the clinical context, changes.

In some embodiments, each of one or more of the generations of a prediction is performed using a trained machine learning unit having a dimensionality higher than a patient data set input to the trained machine learning unit, and the method generates insertion data for one or more of the dimensions of the trained machine learning unit for which no corresponding data is present in the patient data set. Use of a trained machine learning unit having a higher dimensionality than is typically utilized for the patient data set being made available to it, broadens the range of training data that can be used to train the machine learning unit, thereby increasing accuracy. Some of the benefits of the improved training can be obtained even when the patient data set has a lower dimensionality than that of the trained machine learning unit by generating insertion data to be used in place of missing or not yet available data.

According to an aspect of the invention, there is provided a computer-implemented method of monitoring a patient, comprising: receiving a multi-dimensional patient data set at each of a plurality of different reference times, each dimension of the patient data set storing a value representing a different type of information about the patient; and generating a plurality of predictions of a health trajectory of the patient, each prediction being generated using a trained machine learning model receiving as input a different one of the patient data sets, wherein: the trained machine learning model comprises an ensemble of trained first machine learning units and one or more trained second machine learning units; the one or more trained second machine learning units are trained to predict how accurately each trained first machine learning unit would predict a health trajectory as a function of a range of possible patient data sets received by the trained first machine learning unit; the generation of each prediction of the health trajectory comprises performing the following steps: selecting one of the trained first machine learning units from the ensemble based on predictions by the one or more trained second machine learning units of accuracies of prediction of the health trajectory by the trained first machine learning units using the patient data set as input, and generating the prediction of the health trajectory using the selected trained first machine learning unit.

Thus, a methodology is provided that uses a student-teacher architecture to provide informed switching between different trained machine learning units. In contrast to alternative approaches that use a fixed trained machine learning model to make predictions at a fixed point in time, the current approach has been found to provide improved flexibility, accuracy and reliability.

According to an aspect of the invention, there is provided an apparatus for monitoring a patient, comprising: a data receiving unit configured to receive a multi-dimensional patient data set at each of a plurality of different reference times, each dimension of the patient data set storing a value representing a different type of information about the patient; and a data processing unit configured to: generate a plurality of predictions of a health trajectory of the patient, each prediction being generated using a trained machine learning model receiving as input a different one of the patient data sets, wherein the trained machine learning model is dimensionally adaptive, such that predictions of the patient trajectories are provided using patient data sets having different respective dimensionalities for at least a sub-set of the reference times.

According to an aspect of the invention, there is provided an apparatus for monitoring a patient, comprising: a data receiving unit configured to receive a multi-dimensional patient data set at each of a plurality of different reference times, each dimension of the patient data set storing a value representing a different type of information about the patient; and a data processing unit configured to: generate a plurality of predictions of a health trajectory of the patient, each prediction being generated using a trained machine learning model receiving as input a different one of the patient data sets, wherein: the trained machine learning model comprises an ensemble of trained first machine learning units and one or more trained second machine learning units; the one or more trained second machine learning units are trained to predict how accurately each first machine learning unit would predict a health trajectory as a function of a range of possible patient data sets received by the trained first machine learning unit; the generation of each prediction of the health trajectory comprises performing the following steps: selecting one of the trained first machine learning units from the ensemble based on predictions by the one or more trained second machine learning units of accuracies of prediction of the health trajectory by the trained first machine learning units using the patient data set as input, and generating the prediction of the health trajectory using the selected trained first machine learning unit.

Embodiments of the invention will now be described, by way of example only, with reference to the accompanying drawings in which corresponding reference symbols indicate corresponding parts, and in which:

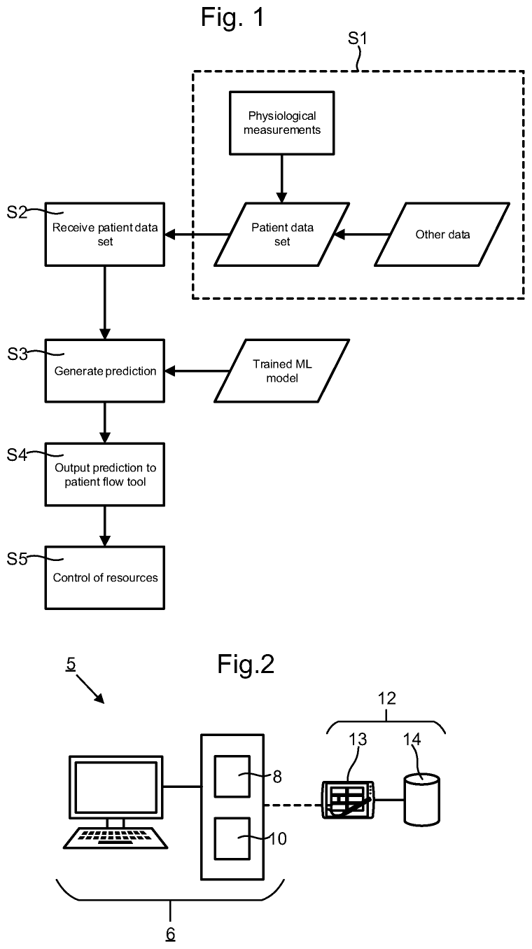

FIG. 1 schematically depicts a method of monitoring a patient according to an embodiment;

FIG. 2 depicts an apparatus for implementing methods of the type depicted in FIG. 1;

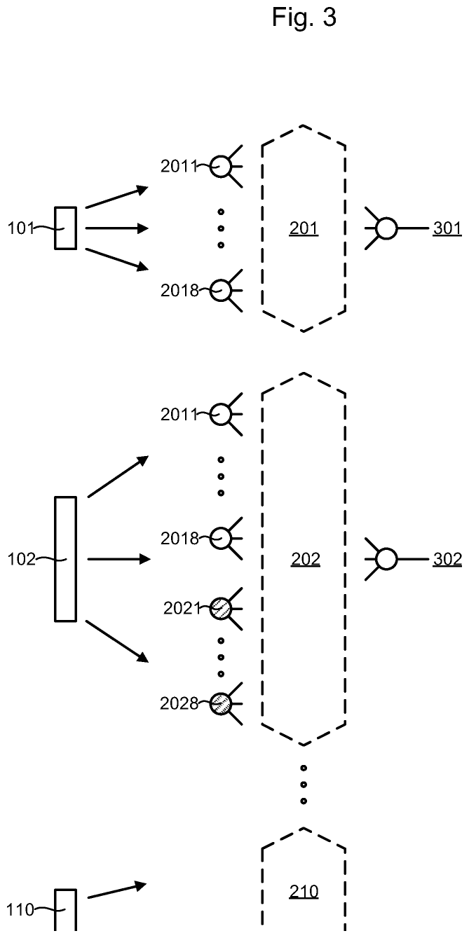

FIG. 3 illustrates selecting different trained machine learning units from an ensemble at different reference times;

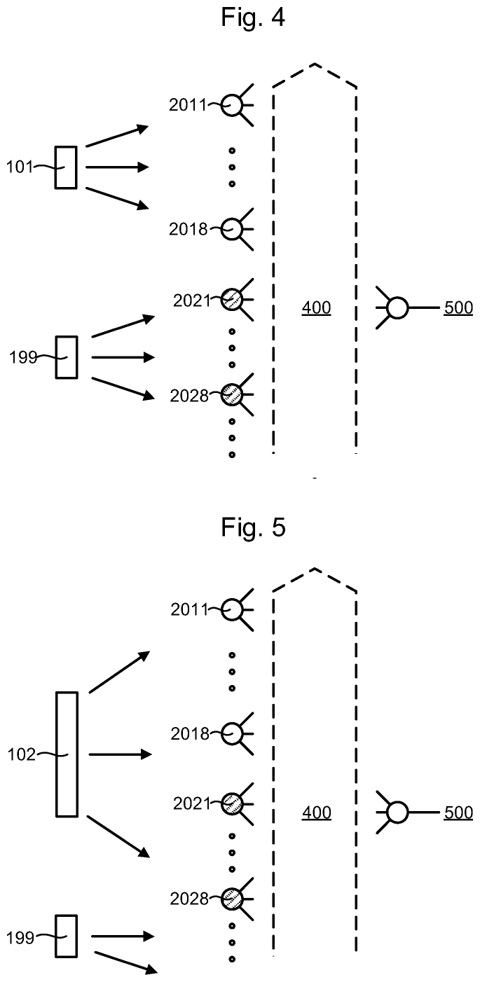

FIGS. 4 and 5 illustrate use of a trained machine learning unit of higher dimensionality at different reference times;

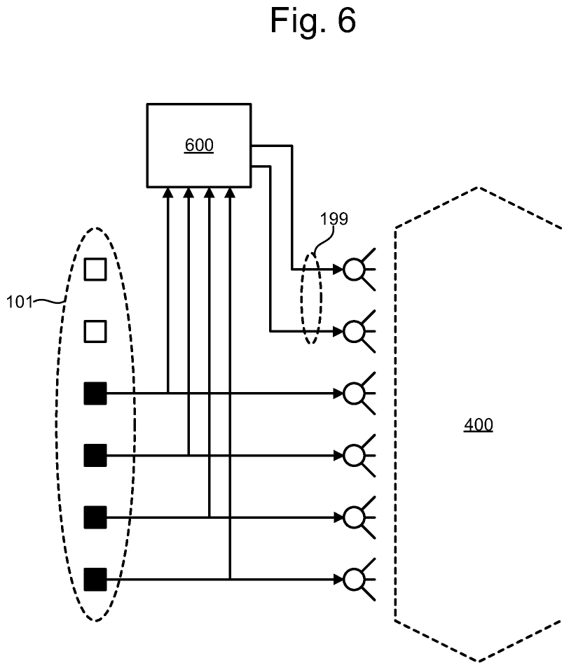

FIG. 6 illustrates use of a patient data set to generate insertion data for use with a trained machine learning unit of the type depicted in FIGS. 4 and 5;

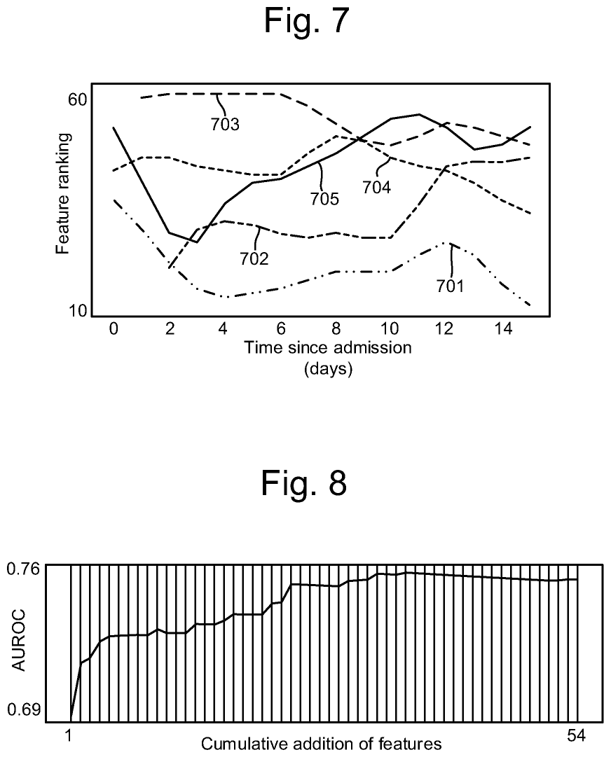

FIG. 7 is a graph showing variation in time in the relative importance of five exemplary features in predicting patient discharge from hospital in 24 hours;

FIG. 8 is a graph showing AUROC score for patient discharge prediction for patients admitted into hospital that day, as a function of incorporating more features into a machine learning unit;

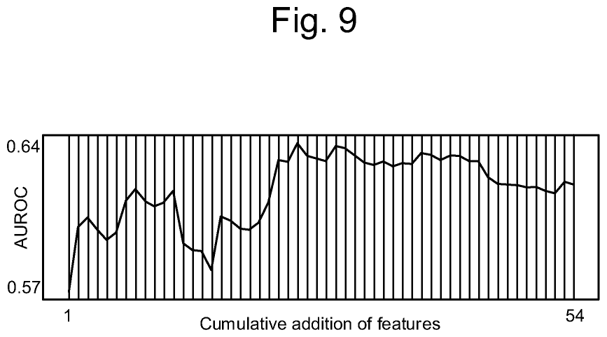

FIG. 9 is a graph corresponding to the graph of FIG. 8 for the same patients 13 days after the admission into hospital;

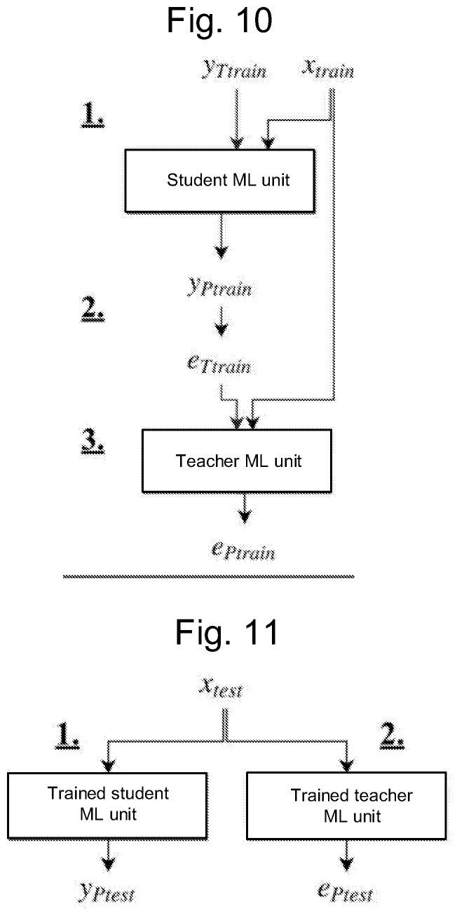

FIGS. 10 and 11 depict an example process of training and using a student-teacher architecture.

Methods of the present disclosure are computer-implemented. Each step of the disclosed methods may therefore be performed by a computer. The computer may comprise various combinations of computer hardware, including for example CPUs, RAM, SSDs, motherboards, network connections, firmware, software, and/or other elements known in the art that allow the computer hardware to perform the required computing operations. The required computing operations may be defined by one or more computer programs. The one or more computer programs may be provided in the form of media, optionally non-transitory media, storing computer readable instructions. When the computer readable instructions are read by the computer, the computer performs the required method steps. The computer may consist of a self-contained unit, such as a general-purpose desktop computer, laptop, tablet, mobile telephone, smart device (e.g. smart TV), etc. Alternatively, the computer may consist of a distributed computing system having plural different computers connected to each other via a network such as the internet or an intranet.

FIG. 1 schematically depicts in flow chart form an example framework for a method of monitoring a patient according to embodiments of the present disclosure. The method may be performed by an apparatus 5 as depicted in FIG. 2.

In step S1, a patient data set is generated. The patient data set is multi-dimensional. The patient data set comprises a value representing each of a plurality of different types of information. Each different type of information defines a different respective one of the dimensions of the patient data set. For example, if ten different types of information are represented by the patient data set, the dimensionality of the patient data set is ten. The patient data set may be represented as a vector.

The information in the patient data set comprises information that is potentially relevant to predicting a health trajectory of a patient. In an embodiment, the patient data set comprises at least physiological data about the patient. The physiological data may be derived from physiological measurements, as indicated in FIG. 1. The physiological measurements may be performed on the patient using a sensor system 12, as depicted in FIG. 2. The sensor system 12 may comprise a local electronic unit 13 (e.g. a tablet computer, smart phone, smart watch, etc.) and a sensor unit 14 (e.g. a blood pressure monitor, heart rate monitor, etc.). In an embodiment, the physiological data comprises one or more of the following: heart rate, respiratory rate, temperature, blood oxygenation, systolic blood pressure, diastolic blood pressure, electrocardiogram, blood glucose, temperature, blood constituent levels, pupil size, pain score, Glasgow coma score or any measurements performed on a sample from the human or animal The patient data set may comprise one or more dimensions representing data other than direct physiological data, e.g. clinical notes, (depicted as “Other data” in FIG. 1).

The patient data set may comprise one or more items of information from an Electronic Patient Record (EHR). An EHR is an electronic version of the traditional paper record which stores patient-based information in the hospital. It typically includes clinical, demographic and other information for all patients admitted to the hospital. The EHR may be used to train machine learning units used in the method (e.g. in step S3 discussed below).

Embodiments of the present disclosure can be implemented (and were tested) using hospital data which included patient demographics, timestamped vital sign measurements, laboratory test results, procedures, diagnosis codes, and a range of other information. The data for each section of a patient's record, for example vital signs recordings or admissions data, are stored in separate tables which can be searched and cross-referenced using the ID of a patient's unique admission.

In some embodiments, the patient data set will additionally comprise information about operational conditions in the medical facility in which the patient is located, such as a capacity and/or occupancy of one or more wards or departments in the medical facility and/or other metrics that correlate with a degree of load on the medical facility. The information about operation conditions may be derived or estimated. A medical facility under excessive load will be less able to optimally provide future treatment in a timely fashion, which can influence the expected health trajectory of the patient.

In step S2, a patient data set is received. In an embodiment, the patient data set is received using a data receiving unit 8 as depicted in FIG. 2. The data receiving unit 8 may form part of a computing system 6 (e.g. laptop computer, desktop computer, etc.). The computing system 6 may further comprise a data processing unit 10 configured to carry out steps of the method.

The patient data set is processed in steps S3-S4 discussed below. In some embodiments, a patient data set is received for each of a plurality of different reference times. The steps S2-S4 are then repeated for each of the patient data sets. Each reference time corresponds to a point in time at which a particular patient data set is available. The patient data set corresponding to a particular reference time contains the most up-to-date information about the patient available at the reference time. The method is thus able to incrementally provide predictions of health trajectory of a patient as more information becomes available. The time interval between separate predictions may depend on one or more of the following: a rate at which new information is made available, a nature of the patient, and a location of the patient in the medical facility (e.g. in an ED or general ward). For example, shorter time intervals may be appropriate for monitoring a patient in the ED (e.g. with intervals of the order of minutes or hours) whereas longer time intervals may be appropriate for monitoring a patient in the general ward (e.g. with intervals of the order of one or more days).

In step S3, a prediction of a health trajectory of the patient is generated. The prediction is generated using a trained machine learning model. The trained machine learning model receives as input the patient data set received in step S2. Where steps S2-S4 are repeated to generate plural predictions, the trained machine learning model will receive as input a different patient data set each time. The trained machine learning model may comprise one or more machine learning units. The machine learning units may be trained at an earlier time (or progressively) so that the trained machine learning model can be stored in a trained form ready for use when needed. The machine learning units may be trained based on various machine learning algorithms, including for example one or more of the following: Logistic Regression Model, Support Vector Machine Model, Decision Tree ensemble methods such as Random Forest Model, Deep Neural Networks such as Multi-Layer Perceptron Model, Recurrent Neural Network and Long Short-Term Memory Models. The training may comprise inputting to the machine leaning unit patient data sets and corresponding health trajectories from a plurality of historical patients.

The health trajectory describes progression of a patient's health state, typically while the patient is in a medical facility. The health trajectory may correlate with a treatment trajectory, and thus with treatments applied to the patient and the times at which the treatments are applied, as well as with physical movements of the patient between different wards or departments in the medical facility (e.g. from an ED to a general ward).

In an embodiment, the prediction of the health trajectory comprises calculating a probability of the patient reaching a reference health state within a predetermined reference period from the prediction. In an embodiment, the reference health state corresponds to a state at which a patient is ready to be transferred out of a reference location in a medical facility, for example out of an ED into a general hospital ward or out of the hospital entirely (i.e. ready for discharge). The prediction of a health trajectory may thus comprise calculation of a probability of the patient being ready for discharge from a medical facility within a predetermined reference period from the generation of the prediction, e.g. within the next 24 hours or within the next 48 hours or within the next 72 hours, etc. Detection of a ready-for-discharge state is an example of detecting a predetermined degree of normality of a patient. The predetermined degree of normality may be represented by a normality score. High normality scores may correspond for example to a patient health status that indicates required care and treatment has been administered and/or that the patient could be ready for discharge within a relatively short time frame (e.g. 24 hours). Low normality scores may correspond for example to a patient health status that indicates further care and treatments may be required, and the patient is not likely to be discharged in the immediate future.

In an alternative class of embodiments, the reference health state corresponds to an adverse health state, such that the prediction of the health trajectory predicts a transition to the adverse health state. In this case, the prediction of the health trajectory may comprise generating a metric related to risk, which may be referred to as a risk score. Generation of such a risk score may be particularly useful for supporting management of an ED in a hospital. High risk scores may correspond to conditions likely to lead to adverse patient outcomes such as death, prolonged hospitalisations with long lengths of stay and so on. Low risk scores may correspond to minor conditions, for which patients can swiftly be treated within and discharged from the ED.

The trained machine learning model used in step S3 is dimensionally adaptive. Predictions of patient trajectories can thus be provided using patient data sets having different respective dimensionalities in different instances of performing steps S1-S4. Examples of how this can be implemented are described below with reference to FIGS. 3-6.

FIG. 3 illustrates an embodiment in which the trained machine learning model switches between different trained machine learning units 201-210 of an ensemble of trained machine learning units 201-210. In the example shown, an ensemble comprising ten trained machine learning units 201-210 is envisaged. In other embodiments, fewer than ten or more than ten trained machine learning units may be provided. In embodiments of this type, the switching between different trained machine learning units 201-210 is controlled based on a dimensionality of each patient data set to be processed. This approach can be implemented with high computational efficiency because each patient data set can be matched with a trained machine learning unit that is adapted to process the patient data set with no or minimal modification of the patient data set, omission of data from the patient data set, and/or generation of insertion data to replace data not present in the patient data set.

In one embodiment using an ensemble of trained machine learning units 201-210, step S3 comprises the following sub-steps.

In a first sub-step, a dimensionality of a received patient data set is determined. In the example of FIG. 3, patient data set 101 is received at a first reference time. The patient data set 101 comprises a vector of eight values, so the dimensionality of the patient data set 101 is eight. At a subsequent, second reference time, patient data set 102 is received (during a subsequent instance of performing steps S1-S4). The patient data set 102 comprises more dimensions than patient data set 101. This may be because more information is now available about the patient, for example due to supplementary tests having been carried out on the patient, and/or training of the trained machine learning model may have indicated that improved prediction is expected at the particular point in time corresponding to input of patient data set 102 if more dimensions are present. In other embodiments, the patient data set 102 may comprise fewer dimensions than patient data set 101, for example due to less information being available and/or training of the trained machine learning model may have indicated that improved prediction is expected at the particular point in time corresponding to input of patient data set 102 if fewer dimensions are present. In either case, the type of information corresponding to each of one or more of the dimensions of the patient data set 102 may be different from the type of information corresponding to the respective dimension in the patient data set 101. Thus, not only may the dimensions of the patient data sets 101 and 102 differ, but the types of information corresponding to at least some the dimensions may also differ. The above flexibility allows the system to adapt to provide optimal predictions where the machine learning importance and availability of different information types varies over time.

In a second sub-step, a trained machine learning unit is selected from the ensemble based on the determined dimensionality of the patient data set 101,102. In an embodiment, the trained machine learning unit is selected such that a dimensionality of the trained machine learning unit is equal to or higher than the dimensionality of the patient data set 101,102. The dimensionality of the trained machine learning unit may be defined by the number of input nodes of the trained machine learning unit (e.g. input nodes of a trained neural network). Thus, in the example of FIG. 3, patient data set 101 having eight dimensions is input to a trained machine learning unit 201, selected from the ensemble, via a corresponding eight input nodes 2011-2018 of the trained machine learning unit 201. The trained machine learning unit 201 is selected because the number of input nodes 2011-2018 equals the dimensionality of the patient data set 101. Patient data set 102 has eight dimensions plus some additional dimensions representing new data about the patient that has become available since a prediction was obtained using the earlier patient data set 101. The higher dimensionality of the patient data set 102 means that the trained machine learning unit 201 would not be able to process all of the information in the patient data set 102. In this case a different trained machine learning unit 202 is selected. Trained machine learning unit 202 has a higher dimensionality than trained machine learning unit 201, with a corresponding larger number of input nodes. In the example shown, the trained machine learning unit 202 has eight input nodes 2011-2018 corresponding to the input nodes 2011-2018 of the trained machine learning unit 201 plus additional input nodes 2021-2028. A total of 18 input nodes are provided by the trained machine learning unit 202, thus enabling the trained machine learning unit 202 to deal with patient data sets, such as patient data set 102, having higher dimensionality than patient data set 101.

In a third sub-step, a prediction of a health trajectory is generated using the selected machine learning unit. The predicted health trajectory may be output via an output node of the trained machine learning unit. In the example of FIG. 3, the predicted health trajectory generated using the patient data set 101 and trained machine learning unit 201 is output from an output node 301 of the trained machine learning unit 201. The predicted health trajectory generated using the patient data set 102 (e.g. at a later time) and trained machine learning unit 202 is output from an output node 302 of the trained machine learning unit 202.

FIGS. 4 and 5 illustrate an embodiment in which the trained machine learning model of step S3 uses the same trained machine learning unit 400 for multiple different patient data sets (in FIG. 4 patient data set 101, and in FIG. 5 patient data set 102) having different dimensionalities relative to each other. This approach may advantageously allow a wider range of data to be used to train the machine learning unit 400 than may be possible using an ensemble of more specific trained machine learning units (such as described above with reference to FIG. 3), which may each need to be trained using relatively specific training data, which may not be so readily available. In this embodiment, the trained machine learning unit 400 is configured to receive an input data set having a higher dimensionality than at least one of the patient data sets 101,102 that are to be processed. For example, the number of input nodes of the trained machine learning unit 400 may be equal to or larger than the dimensionality of the largest patient data set that it is envisaged the trained machine learning unit 400 will need to process. FIG. 4 illustrates input of a patient data set 101 having eight dimensions to a corresponding eight input nodes 2011-2018 of the trained machine learning unit 400. The trained machine learning unit 400 has further input nodes, including 2021-2028 and others. These further input nodes do not correspond to specific dimensions of the patient data set 101. The trained machine learning unit 400 would, however, have been trained with data supplied to all of its input nodes and would normally be expected to provide the most accurate predictions when input is provided to all available input nodes. In embodiments of this type, the method may thus further comprise generating insertion data 199 for one or more of the dimensions of the trained machine learning unit 400 for which no corresponding data is present in the patient data set 101 being processed. The insertion data 199 may be generated (e.g. imputed or inferred) in various ways, including for example via statistical analyses of historical data for the same patient or for one or more cohorts of patients of similar type. An example process is described in further detail with reference to FIG. 6 below. FIG. 5 illustrates input of a patient data set 102 having more than eight dimensions to a corresponding number of input nodes 2011-2018 and 2021-2028 of the trained machine learning unit 400. Again, insertion data 199 may be generated to supply input data to one or more of the other input nodes of the trained machine learning unit 400. The predicted health trajectory is output in each case from an output node 500 of the trained machine learning unit 400.

In an embodiment, as depicted in FIG. 6, the generation of the insertion data 199 is performed at least partly by using the patient data set 101. In the example shown, the patient data set 101 comprises a vector of four values (i.e. has a dimensionality of four), indicated by the solid square elements. Two dimensions are not present (or contain missing or unreliable values), as indicated by the open square elements in the patient data set 101. An insertion data generation module 600 (implemented by suitable computer hardware and/or software, for example) receives input from the patient data 101, as depicted by the arrows leading to the insertion data generation module 600, and uses the input to generate insertion data 199. The insertion data 199 is input to the trained machine learning unit 400 such that the trained machine learning unit receives input data at each and every one of the input nodes of the trained machine learning unit. In an embodiment, the generation of the insertion data 199 is performed by using the patient data set 101 to assign the patient to one of a plurality of predetermined patient groups and predicting one or more values for the insertion data 199 based on historical data for the patient group to which the patient has been assigned. The predetermined patient groups may correspond to patient groups having common physiological characteristics or common risk scores, for example.

In some embodiments, switching between machine learning units 201-210 can occur exclusively as a function of time. That is, the switching can be informed by where a patient is in their respective care journey pathway, whether that is well defined time points like patients' day of stay, or variable passages of time that are instead defined by the patients' stage of care (on admission, on triage and so on). In both cases additional information often becomes available as the patient progresses through their care pathway, or existing information takes on different importance and value. A machine learning model selecting between machine learning units 201-210 trained specifically for these contexts can be expected to achieve improved performance relative to prior art approaches, as described above.

Whilst improved performance can be expected on average by means of the dimensional adaption described above, for a subset of patients it may be that predictive performance decreases or could be improved more optimally. Embodiments are described below that address this challenge using a “student-teacher” architecture, a method in which an additional “teacher” machine learning unit (referred to below as a trained second machine learning unit) is trained to predict the error that a “student” machine learning unit (referred to below as a trained first machine learning unit) is expected to make in its individual predictions. In a simple application, the prediction made by the teacher can inform a user which of the student's predictions can and cannot be trusted. This in turn can inform which of the trained student units are most reliable for the patient in question, and whether updated predictions for an individual patient should be considered by switching to a different student unit.

In the embodiment discussed above with reference to FIG. 3, a trained machine learning model switches between different trained machine learning units 201-210 of an ensemble of trained machine learning units 201-210 based on a dimensionality of each patient data set to be processed. This approach can be adapted to use the student-teacher architecture mentioned above as follows.

In an embodiment, the ensemble of trained machine learning units 201-210 may be referred to as an ensemble of trained first machine learning units 201-210 (each unit being an example of a student machine learning unit). The trained machine learning model may then further comprise one or more trained second machine learning units (not shown in FIG. 3). Optionally, a trained second machine learning unit may be provided for each trained first machine learning unit 201-210. Each trained second machine learning unit is an example of a teacher machine learning unit. The one or more trained second machine learning units are trained to predict how accurately each trained first machine learning unit would predict a health trajectory as a function of a range of possible patient data sets received by the trained first machine learning unit 201-210. The first and second machine learning units may be trained based on various (optionally different) machine learning algorithms, including for example one or more of the following: Logistic Regression Model, Support Vector Machine Model, Decision Tree ensemble methods such as Random Forest Model, Deep Neural Networks such as Multi-Layer Perceptron Model, Recurrent Neural Network and Long Short-Term Memory Models.

In embodiments of this type, the switching between different trained machine learning units may comprise switching between different ones of the trained first machine learning units 201-210 based on the dimensionality of the patient data set (as described above with reference to FIG. 3) and an output from the one or more trained second machine learning units providing predicted accuracies of the trained first machine learning units in respect of the patient data set (i.e. using the student-teacher architecture to predict accuracies of prediction when the given patient data set is used by the student as the basis for prediction).

Alternatively, the switching may be performed without necessarily using the dimensionality. In an embodiment of this type, the following steps may be performed. A patient data set may be generated and received as described above with reference to steps S1 and S2 of FIG. 1. A plurality of predictions of a health trajectory of the patient are generated, each prediction being generated using a trained machine learning model receiving as input a different one of the patient data sets. The trained machine learning model comprises an ensemble of trained first machine learning units 201-210 and one or more trained second machine learning units. The one or more trained second machine learning units are trained to predict how accurately each trained first machine learning unit 201-210 would predict a health trajectory as a function of a range of possible patient data sets received by the trained first machine learning unit 201-210. In step S3 of FIG. 1, the generation of each prediction of the health trajectory is performed by the machine learning model performing the following steps: 1) selecting one of the trained first machine learning units 201-210 from the ensemble based on predictions by the one or more trained second machine learning units of accuracies of prediction of the health trajectory by the trained first machine learning units 201-210 using the patient data set as input (e.g. selecting the trained first machine learning unit having the highest predicted accuracy for that particular patient data set); and 2) generating the prediction of the health trajectory using the selected trained first machine learning unit 201-210.

Exemplary further implementation details for a student-teacher architecture are given below.

A trained first machine learning unit 201-210 may be trained to make predictions for a desired application given the feature set available to it (e.g. will a patient be discharged from hospital in the next 24 hours given their demographic, physiological information etc.). The first machine learning unit 201-210 may be trained retrospectively on past historical data using existing machine learning algorithms such as neural networks, SVM and so on (as described above). An example process of training and using a student-teacher architecture is shown graphically in FIGS. 10 and 11 and described as follows.

In the case of a binary classification problem (such as patient discharge prediction), we let 0≤y_{P}≤1 represent the classification probability score output by the trained first machine learning unit (student) where y_{P}<0.5 represents a negative class prediction and y_{P}>0.5 denotes a positive class prediction. The true class of a sample is given by y_{T} {0, 1} and the error of a classification made by the trained first machine learning unit (student) is defined as e=|y_{T}−y_{P}|. For example, if the trained first machine learning unit (student) assigns a probability classification of y_{P}=0.3 to a patient, the trained first machine learning unit (student) is predicting that the patient will not be discharged. If the true class of the patient is y_{T}=1, this means that the patient was in fact discharged and the prediction error is e=0.7. However, if the true class of the patient had been y_{T}=0, the prediction error would be e=0.3.

The features, as input to the trained first machine learning unit (student), are represented by xₜᵣₐᵢₙ and xₜₑₛₜ for training and test datasets respectively. The true class of samples in the training and test datasets, often known as ‘labels’ in the context of classification, are denoted y_{Ttrain} and y_{Ttest} respectively, and the classes predicted by the trained first machine learning unit (student) are given by y_{Ptrain} and y_{Ptest}. The true error of these predicted classifications are denoted by e_{Ttrain} and e_{Ttest} for the two datasets, while the predicted errors, as output by the trained second machine learning unit (teacher) are denoted e_{Ptrain} and e_{Ptest}. The process of training and testing the student-teacher architecture for each first machine learning unit (student) may be as follows:
1. The first machine learning unit (student) is trained using xₜᵣₐᵢₙ to learn to classify y_{Ttrain}.
2. The performance of the trained first machine learning unit (student) on xₜᵣₐᵢₙ is evaluated, with its output y_{Ptrain} used to calculate e_{Ttrain}.
3. Using the same features as were used to train the first machine learning unit (student), xₜᵣₐᵢₙ, the second machine learning unit (teacher) is trained to predict the classification error e_{Ttrain}. The output of the trained second machine learning unit (teacher) is e_{Ptrain} when its input is xₜᵣₐᵢₙ. The second machine learning unit (teacher) can be trained to minimize the absolute difference between e_{Ttrain} and e_{Ptrain} or any alternatively defined scheme (log of the absolute difference and so on).
   Once trained, the networks can be used to make predictions, via the following steps:
1. The unseen test dataset xₜₑₛₜ is fed to the trained first machine learning unit (student) which outputs y_{Ptest}.
2. The unseen test dataset xₜₑₛₜ is fed to the trained second machine learning unit (teacher) which outputs e_{Ptest}.
3. The predicted error e_{Ptest} is used to inform clinicians which classifications y_{Ptest} can and cannot be trusted, with a high predicted error indicating that the discharge classification made by the first machine learning unit (student) cannot be trusted, and in the context of switching between first machine learning units (students), this one should not be considered (or if it is, it should be with caution).

The accuracy of the trained second machine learning unit (teacher) at predicting the trained first machine learning unit's (student's) error can additionally inform the level of caution associated with the trained first machine learning unit's (student's) prediction. The first machine learning unit (student) can be either a classifier or a regressor, the principles of the student-teacher architecture remain the same. It is worth noting that the teacher and student units are independent designs and can be based on different ML algorithms (e.g. the student unit may employ an SVM algorithm while the teacher unit employs a neural network)

By developing and training individual second machine learning units (teachers) for each trained first machine learning unit (student), the student-teacher architecture gives a probabilistic view of a prediction rather than just a score. It thus allows a level of confidence in the predictions made to be ascertained. In the context of this application, this can inform which of the available trained first machine learning units are best to switch to for a given patient. Thus improving overall predictive performance by allowing informed switching.

In step S4, the prediction generated in step S3 is output to a patient flow tool, implemented by a computer for example. The patient flow tool may be configured to support control of resources in a medical facility in step S5. The control of resources may be implemented partially or completely by the patient flow tool itself, or the patient flow tool may organise and/or display information to a user that allows the user to take steps to control resources via other means. In one embodiment, the patient flow tool uses one or more predictions of a health trajectory to support decision making (optionally implemented by a computer) about whether and when to transfer a patient out of a reference location in a medical facility (e.g. to discharge a patient from a hospital). In one embodiment of this type, the patient flow tool uses the generated predictions to rank patients according to how likely they are to be discharged within a predetermined reference period (e.g. in the next 24 hours). In an embodiment, resources (e.g. medical worker time) are allocated preferentially towards patients that are higher ranked, thereby facilitating earlier discharge of patients that are ready for discharge and promoting efficient ward occupancy.

### DETAILED EXAMPLES & RESULTS

### Hospital Discharge Prediction at Different Points in Time

For the task of predicting whether a patient will be discharged from hospital within the next 24 hours, FIG. 7 illustrates that depending on what stage that patient is in their journey (i.e. whether they were admitted that day, or they have already been in the hospital for two weeks), the same feature (i.e. a type of information corresponding to one dimension of a patient data set) may hold different machine learning value. In the particular example of FIG. 7, the variation of a relative ranking (in terms of machine learning value) as a function of time is shown for the following features: whether the day of the week is Friday (curve 701), the time elapsed since a last procedure performed on the patient (curve 705), the average early warning score of the patient between 0-24 hours of their admission (curve 703), the variation of the early warning score between 48-72 hours of their admission (curve 702) and whether the patient is currently in the Intensive Care Unit (ICU) (curve 704). Significant variations in the machine learning value of each type of information is seen as a function of time, with some becoming increasingly important (e.g. curves 702, 704 and 705) and others becoming less important (e.g. curves 701 and 703). The inventors have found that improved predictions can be obtained by configuring the trained machine learning model so that it can switch between different trained machine learning units as a function of time, so as to optimize use of information that is available and its relative machine learning value. For example, based on the variations shown in FIG. 7, it might be expected that progressing from a machine learning unit configured to receive patient data with dimensions corresponding to the types of information of curves 701, 703, 704 and 705 to a machine learning unit configured to receive patient data with dimensions corresponding to the types of information of curves 702, 704 and 705 (i.e. with lower dimensionality in this case) may improve the predictive performance of the overall trained machine learning model.

The above principles are further supported by the graphs of FIGS. 8 and 9, which illustrate how different feature sets provide optimal prediction at different points in time. FIG. 8 provides data corresponding to a first day of admission. FIG. 9 provides data corresponding to 13 days into a patient's hospital stay. The vertical axis in FIGS. 8 and 9 represents the Area Under the Receiver Operating Characteristic curve (AUROC). The AUROC is an important and widely used metric for evaluating the performance of a classifier. The ROC curve of a classifier plots the true positive rate against the false positive rate of said classifier at different discrimination thresholds. The AUROC computes the integral of this curve, producing values ranging from 0 to 1. A value closer to 1 indicates a classifier that is able to correctly classify a higher proportion of true positives before incorrectly classifying a negative class as positive. A high AUROC thus indicates a classifier with strong diagnostic capability

FIGS. 8 and 9 show how the AUROC varies for the two different time points (day 1 and day 13 respectively) as features represented as numbers along the horizontal axis are cumulatively added as training inputs to train a machine learning unit. Thus, for example, at position 4 on the horizontal axis, the AUROC (vertical axis) represents the predictive performance of a machine learning unit trained using features 1-4 only. At position 54 on the horizontal axis, the AUROC represents the predictive performance of a machine learning unit trained using all 54 of the available features.

The features do not appear in the same order in FIGS. 8 and 9 because for different points in a patient's journey (1st vs 13th day of stay) a feature can have radically different machine learning value. Features are listed below in the same order as they appear in FIG. 8, followed by the index position they have in FIG. 9 in square brackets.

Feature 1 [1]=Mean length of stay for historical patients with the same Clinical Classification Software (CCS) code, that is in the same broad diagnostic category.

Feature 2 [34]=Mean National Early Warning Score (NEWS) for patient's admission.

Feature 3 [6]=Mean NEWS for patient between 0-24 hours of their admission.

Feature 4 [5]=Standard deviation of length of stay for historical patients with the same CCS code.

Feature 5 [7]=Most recent NEWS for patient.

Feature 6 [3]=Maximum NEWS for patient between 0-24 hours of their admission.

Feature 7 [21]=Minimum NEWS for patient between 0-24 hours of their admission.

Feature 8 [42]=Minimum NEWS for patient since admission.

Feature 9 [35]=Maximum NEWS for patient since admission.

Feature 10 [54]=Patient age.

Feature 11 [29]=Charlson Co-morbidity Index (CCI) a score of likely mortality given the type and number of existing comorbidities a patient has.

Feature 12 [12]=Variance of NEWS for patient between 0-24 hours of their admission.

Feature 13 [32]=First NEWS for patient on admission.

Feature 14 [46]=Number of ICU admissions.

Feature 15 [22]=Is the patient currently in the ICU.

Feature 16 [43]=Variance of NEWS for patient since admission.

Feature 17 [9]=Time elapsed since patient discharged from operating theatre.

Feature 18 [13]=Time elapsed since patient's last procedure.

Feature 19 [51]=Time elapsed since patient admitted into ICU.

Feature 20 [36]=Patient has had a surgical ICU admission.

Feature 21 [37]=Number of theatre visits.

Feature 22 [53]=If the patient admission was elective or emergency.

Feature 23 [20]=Has the patient been to the operating theatre?

Feature 24 [8]=Number of vital sign observations made between 0-24 hours of patient's admission.

Feature 25 [50]=Patient has had a cardiac ICU admission.

Feature 26 [41]=Patient has had a CTTC ICU admission.

Feature 27 [47]=Was the patient's ICU admission planned?

Feature 28 [48]=Was the patient's ICU admission unplanned?

Feature 29 [49]=Patient has had a AICU ICU admission.

Feature 30 [45]=Had the patient had a procedure during their hospital stay?

Feature 31 [17]=Is today Sunday?

Feature 32 [40]=Is today Friday?

Feature 33 [2]=Is today Saturday?

Feature 34 [52]=Patient has had a non surgical ICU admission.

Feature 35 [44]=Difference between NEWS for a patient on admission and the most recent NEWS for said patient.

Feature 36 [28]=Number of procedures carried out on the patient.

Feature 37 [39]=Is today Monday?

Feature 38 [23]=Is today Thursday?

Feature 39 [25]=Is today Wednesday?

Feature 40 [24]=Is today Tuesday?

Feature 41 [38]=Time elapsed since patient discharged from ICU.

Feature 42** [4]=Maximum NEWS for patient between 48-72 hours of their admission.

Feature 43** [26]=Mean NEWS for patient between 72-96 hours of their admission.

Feature 44** [31]=Maximum NEWS for patient between 72-96 hours of their admission.

Feature 45** [33]=Minimum NEWS for patient between 72-96 hours of their admission.

Feature 46** [10]=Mean NEWS for patient between 48-72 hours of their admission.

Feature 47** [18]=Minimum NEWS for patient between 48-72 hours of their admission.

Feature 48** [15]=Variance of NEWS for patient between 48-72 hours of their admission.

Feature 49** [14]=Number of vital sign observations made between 48-72 hours of patient's admission.

Feature 50** [16]=Mean NEWS for patient between 24-48 hours of their admission.

Feature 51** [30]=Minimum NEWS for patient between 24-48 hours of their admission.

Feature 52** [27]=Variance of NEWS for patient between 24-48 hours of their admission.

Feature 53** [11]=Number of vital sign observations made between 24-48 hours of patient's admission.

Feature 54** [19]=Maximum NEWS for patient between 24-48 hours of their admission.

**: features 42-54 (NEWS on days 1 onwards) are clearly not available for predictions on day 1 (FIG. 8), and are just fed into the model as scores of 0.

FIG. 8 shows an AUROC that increases roughly monotonically as features are added, indicating that good predictive performance is achieved if the machine learning unit corresponding to time=day 1 is trained using all 54 features. In contrast, FIG. 9 shows that a peak in AUROC is achieved towards feature 25, suggesting that a machine learning unit corresponding to time=day 13 would provide better performance if only the first 25 features were used for training (with features 26 onwards not being included).

### Emergency Department (ED) Patient Health Status Prediction at Different Points in Time

Preliminary results for ED patient risk classification, where not being seen and treated within 4 hours in the ED and eventually being hospitalised is used as a proxy for high risk classification, shows that at point of admission the Area Under the Receiver Operating Characteristic curve (AUROC) of an SVM classifier is 0.83, whilst at point of triage (upon incorporating more information), the AUROC increases to 0.87.

At point of admission into the ED, features consist of administrative information (day of the week, calendar month, time of day and so on), demographic information related to the patient (age, gender and so on), weather and climate information (average temperature, number of sunlight hours and so on), ED operational information as well as short-term operational history (current capacity, number of attendances in the last 12 hours and so on) and some clinical information (whether the patient arrived by ambulance or not, whether they have recently been admitted into an ED and so on). A total of 32 features were considered at this point.

At point of triage, an additional 18 features were considered. These related to medical information such as an early warning score, vital sign information, the tests ordered and so on. The increase in AUROC highlights the improvement obtained in risk prediction by including more informative features as they become available through the course of a patient's stay

1. A computer-implemented method of monitoring a patient, comprising:
receiving a multi-dimensional patient data set at each of a plurality of different reference times, each dimension of the patient data set storing a value representing a different type of information about the patient; and
generating a plurality of predictions of a health trajectory of the patient, each prediction being generated using a trained machine learning model receiving as input a different one of the patient data sets,
wherein the trained machine learning model is dimensionally adaptive, such that predictions of the patient trajectories are provided using patient data sets having different respective dimensionalities for at least a sub-set of the reference times.

2. The method of claim 1, wherein the trained machine learning model switches between different trained machine learning units of an ensemble of trained machine learning units, the switching being controlled for each patient data set based on a dimensionality of the patient data set.

3. The method of claim 2, wherein the generation of each prediction comprises:
determining a dimensionality of the patient data set;
selecting a trained machine learning unit from the ensemble based on the determined dimensionality of the patient data set; and
generating the prediction of the health trajectory using the selected trained machine learning unit.

4. The method of claim 1, wherein each of one or more of the generations of a prediction is performed using a trained machine learning unit having a dimensionality higher than a patient data set input to the trained machine learning unit.

5. The method of claim 4, further comprising generating insertion data for one or more of the dimensions of the trained machine learning unit for which no corresponding data is present in the patient data set.

6. The method of claim 5, wherein the generation of the insertion data is performed using the patient data set.

7. The method of claim 6, wherein the generation of the insertion data is performed by using the patient data set to assign the patient to one of a plurality of predetermined patient groups and predicting one or more values for the insertion data using historical data for the patient group to which the patient has been assigned.

8. The method of claim 2, wherein:
the ensemble of trained machine learning units comprises an ensemble of trained first machine learning units and the trained machine learning model further comprises one or more trained second machine learning units;
the one or more trained second machine learning units are trained to predict how accurately each trained first machine learning unit would predict a health trajectory as a function of a range of possible patient data sets received by the trained first machine learning unit; and
the switching between different trained machine learning units comprises switching between different ones of the trained first machine learning units based on the dimensionality of the patient data set and an output from the one or more trained second machine learning units providing predicted accuracies of the trained first machine learning units in respect of the patient data set.

9. A computer-implemented method of monitoring a patient, comprising:
receiving a multi-dimensional patient data set at each of a plurality of different reference times, each dimension of the patient data set storing a value representing a different type of information about the patient; and
generating a plurality of predictions of a health trajectory of the patient, each prediction being generated using a trained machine learning model receiving as input a different one of the patient data sets, wherein:
the trained machine learning model comprises an ensemble of trained first machine learning units and one or more trained second machine learning units;
the one or more trained second machine learning units are trained to predict how accurately each trained first machine learning unit would predict a health trajectory as a function of a range of possible patient data sets received by the trained first machine learning unit;
the generation of each prediction of the health trajectory comprises performing the following steps:
selecting one of the trained first machine learning units from the ensemble based on predictions by the one or more trained second machine learning units of accuracies of prediction of the health trajectory by the trained first machine learning units using the patient data set as input, and
generating the prediction of the health trajectory using the selected trained first machine learning unit.

10. The method of claim 1, wherein each prediction of the health trajectory comprises calculating a probability of the patient reaching a reference health state within a predetermined reference period.

11. The method of claim 10, wherein the reference health state corresponds to a state at which the patient is ready to be transferred out of a reference location in a medical facility.

12. The method of claim 11, wherein the reference health state corresponds to a state at which the patient is ready for discharge from the medical facility.

13. The method of claim 1, wherein each patient data set comprises at least physiological data about the patient.

14. The method of claim 13, wherein the physiological data comprises data derived from measurements performed on the patient using a sensor system.

15. The method of claim 14, wherein the physiological data comprises one or more of the following: heart rate, respiratory rate, temperature, blood oxygenation, systolic blood pressure, diastolic blood pressure, electrocardiogram, blood glucose, temperature, blood constituent levels, pupil size, pain score, Glasgow coma score or any measurements performed on a sample from the human or animal.

16. The method of 14, further comprising performing physiological measurements to generate the physiological data.

17. A computer program comprising instructions which, when the program is executed by a computer, cause the computer to carry out the method of claim 1.

18. A computer program product comprising instructions which, when the program is executed by a computer, cause the computer to carry out the method of claim 1.

19. An apparatus for monitoring a patient, comprising:
a data receiving unit configured to receive a multi-dimensional patient data set at each of a plurality of different reference times, each dimension of the patient data set storing a value representing a different type of information about the patient; and
a data processing unit configured to:
generate a plurality of predictions of a health trajectory of the patient, each prediction being generated using a trained machine learning model receiving as input a different one of the patient data sets,
wherein the trained machine learning model is dimensionally adaptive, such that predictions of the patient trajectories are provided using patient data sets having different respective dimensionalities for at least a sub-set of the reference times.

20-21. (canceled)
